(19) United States
(12) Patent Application Publication
(10) Pub. No.: US 2008/0097142 A1
(43) Pub. Date: Apr. 24, 2008

(54) MAGNETIC FIELD GENERATOR, METHOD OF GENERATING A PULSED SINUSOIDAL MAGNETIC WAVE AND MAGNETIC FIELD GENERATOR SYSTEM

(76) Inventor: PAUL SAVAGE, Alachua, FL (US)

Correspondence Address: ECKERT SEAMANS CHERIN & MELLOTT, 600 GRANT STREET, 44TH FLOOR, PITTSBURGH, PA, 15219, US

(21) Appl. No.: 11/871,433

(22) Filed: Oct. 12, 2007

Related U.S. Application Data
(60) Provisional application No. 60/853,222, filed on Oct. 20, 2006.

(51) Int. Cl. A61N 2/04

(52) U.S. Cl. 600/14; 600/15

(57) ABSTRACT

The invention generally relates to a magnetic field generator. The magnetic field generator has a power source and a microcontroller in electrical communication with the power source. A coil is also provided that is in electrical communication with the microcontroller. The coil generates a pulsed magnetic wave of an alternating current when a sensor determines that the magnetic field generator is located in proximity to a living being. The magnetic field generator is structured to be coupled to a device and the device is structured to be worn by the living being in proximity to the living being. A method of generating a pulsed magnetic wave to a device is also provided. A magnetic field generator system is also provided that has a magnetic field generator which is structured to be in communication with a charger/tester.

### PARENT CASE TEXT

This patent application claims priority under 35 USC §119(e)(1) to provisional patent application No. 60/853,222 filed Oct. 20, 2006, the contents of which is hereby incorporated by reference into this patent application in its entirety as if fully set forth herein.

### FIELD OF THE INVENTION

This invention generally relates to a magnetic field generator, a method of generating a pulsed sinusoidal magnetic wave and a magnetic field generator system.

### BACKGROUND OF THE INVENTION

The Earth has a natural electromagnetic frequency range from about 7.3 hertz to about 8.3 hertz. This frequency range is known as the Schumann Resonance electromagnetic frequency range and is also referred to as the Earth's natural electromagnetic frequency.

Due to the proliferation of electronic devices throughout the world, electronic devices have been adding to the ambient frequency of the atmosphere in which living beings exist. The addition to the ambient frequency in the atmosphere causes living beings to suffer, for example, from sickness, anxiety, depression and exhaustion.

Certain scientific studies indicate that exposure of living beings to the Schumann Resonance electromagnetic frequency range promotes a state of calmness and relaxation. Exposure of living beings to the Schumann Resonance electromagnetic frequency range is believed to recalibrate the resonance of living beings suffering from exposure to other frequencies supplied by the worldwide proliferation of electronic devices.

Accordingly, a need exists in the art for a magnetic field generator, a method of generating a pulsed magnetic wave and a magnetic field generator system that emulates the Earth's Schumann Resonance electromagnetic frequency range.

### SUMMARY OF THE INVENTION

An object of the invention is to provide a magnetic field generator that emulates the Earth's Schumann Resonance electromagnetic frequency range.

Another object of the invention is to provide a method of generating a pulsed magnetic wave that emulates the Earth's Schumann Resonance electromagnetic frequency range.

An additional object of the invention is to provide a magnetic field generator system that emulates the Earth's Schumann Resonance electromagnetic frequency range.

A further object of the invention is to provide a magnetic field generator, a method of generating a pulsed magnetic wave and a magnetic field generator system that promotes a state of calmness and relaxation in living beings.

Certain objects of the invention are achieved by providing a magnetic field generator. The magnetic field generator has a power source and a microcontroller in electrical communication with the power source. A coil is also provided that is in electrical communication with the microcontroller. The coil generates a pulsed magnetic wave of an alternating current when a sensor determines that the magnetic field generator is located in proximity to a living being. The magnetic field generator is structured to be coupled to a device and the device is structured to be worn by the living being in proximity to the living being.

Other objects of the invention are achieved by providing a method of generating a pulsed magnetic wave to a device to be worn by a living being. The method comprises monitoring time over a twenty-four hour period, generating a pulsed magnetic wave of a certain frequency about every two seconds to about ten seconds when a sensor determines that the device is located in proximity to the living being and altering the frequency of the pulsed magnetic wave over a twenty-four hour period.

Other objects of the invention are achieved by providing a magnetic field generator system. The magnetic field generator system has a magnetic field generator having a rechargeable power source and a microcontroller in electrical communication with the rechargeable power source. A coil is also provided that is in electrical communication with the microcontroller. The coil generates a pulsed magnetic wave of an alternating current when a sensor determines that the magnetic field generator is located in proximity to a living being. The magnetic field generator is structured to be coupled to a device and the device is structured to be worn by the living being in proximity to the living being. The magnetic field generator is structured to be in communication with a charger/tester.

### BRIEF DESCRIPTION OF THE DRAWINGS

FIG. 1 is a block diagram of a magnetic field generator;

FIGS. 2a and 2b are flow charts showing the operations performed by executable code of the microcontroller provided in the magnetic field generator;

FIG. 3 is a view of a variety of devices worn by a living being that contain a magnetic field generator;

FIG. 4 is a block diagram of the charger/tester;

FIGS. 5a and 5b are flow charts showing the operations performed by executable code of the charger/tester; and

FIG. 6 is a flow chart showing the operations performed by executable code of the microcontroller provided in the charger/tester when the magnetic field generator is being charged.

### DETAILED DESCRIPTION OF THE PREFERRED EMBODIMENTS

As employed herein, the term “number” shall mean one or an integer greater than one (i.e., a plurality). As employed herein, the statement that two or more parts are “attached”, “connected”, “coupled”, or “engaged” together shall mean that the parts are joined together either directly or joined through one or more intermediate parts.

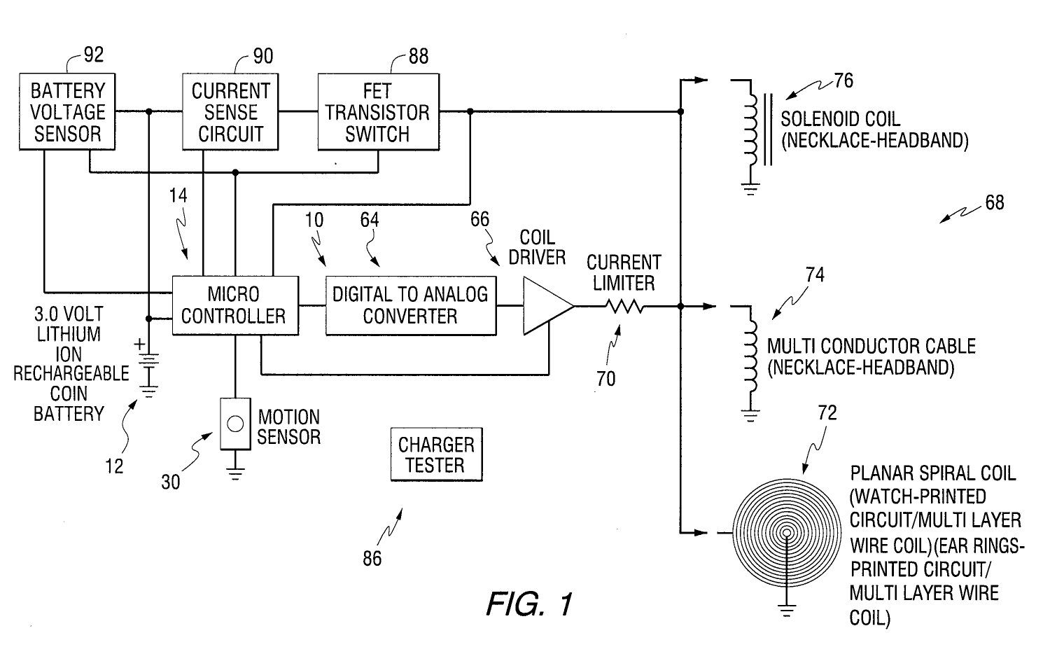

Turning to FIG. 1, a magnetic field generator 10 is shown. The magnetic field generator 10 has a reduced form factor and may be coupled to a device such as, for example, a watch, a pendent, a headband or earrings. The magnetic field generator 10 has a power source 12 coupled to a microcontroller 14. The power source 12 is in electrical communication with the microcontroller 14. The power source 12 is of a low voltage type. As used herein, the term “low voltage” means around three volts or less. As an example, the power source 12 may consist of a commonly available three volt lithium coin type battery which supplies power to the magnetic field generator 10 circuitry. In one embodiment, an end-user may replace the battery in the magnetic field generator 10 when the battery has been consumed in the situation where a non-rechargeable power source 12 is employed. In an another embodiment, an end-user may recharge the battery in the magnetic field generator 10 when the battery has been consumed in the situation where a rechargeable power source 12 is employed.

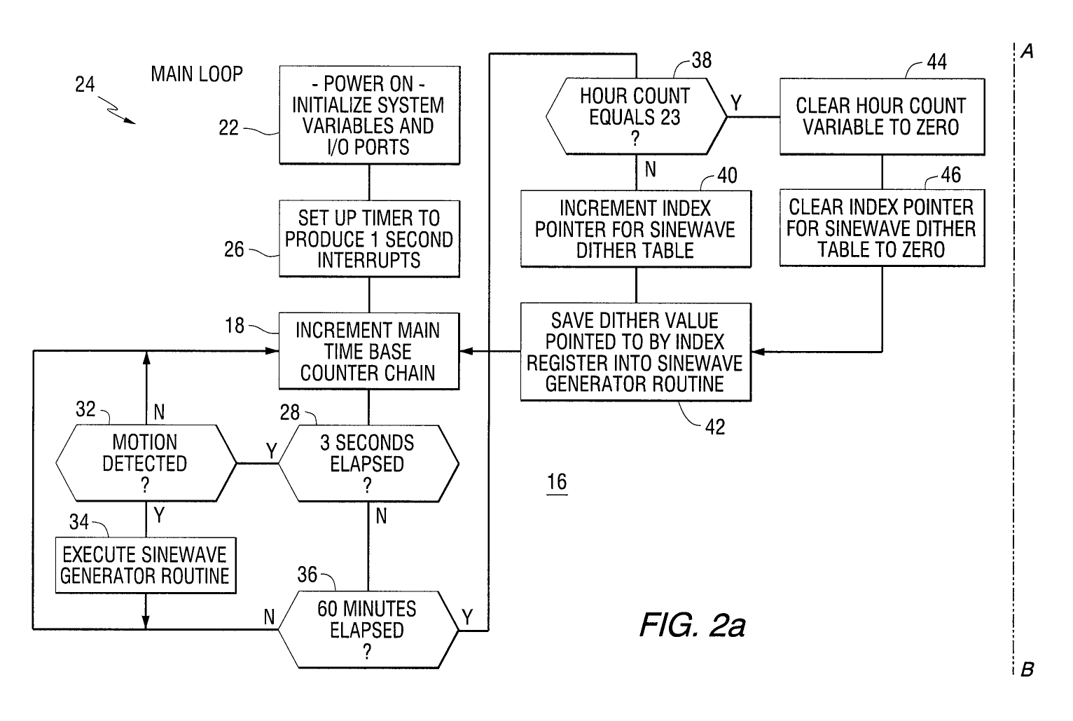

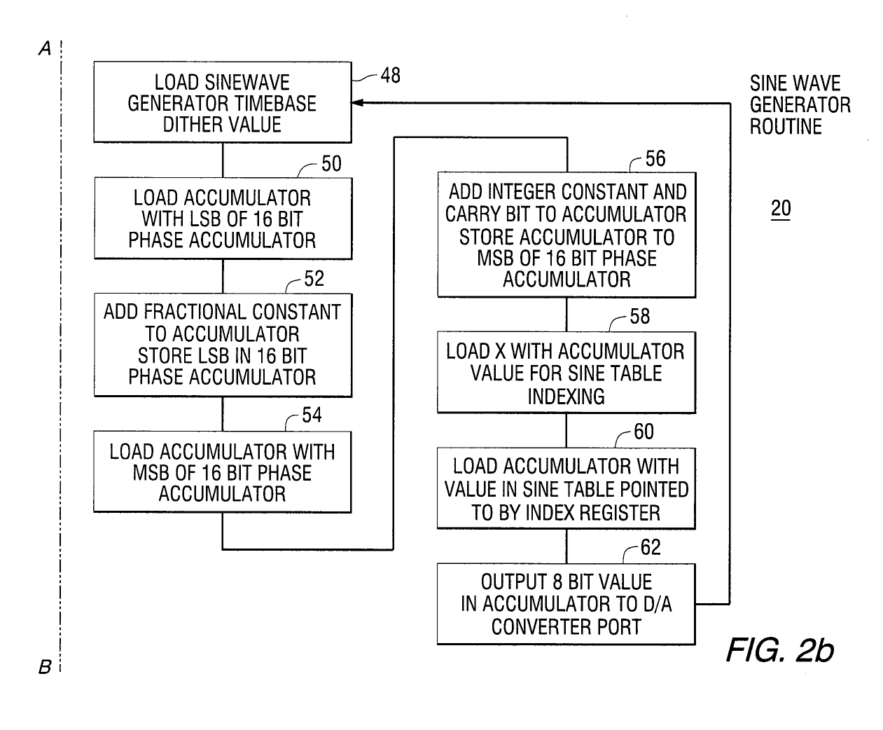

The microcontroller 14 contains executable code which runs a routine 16 as shown in FIG. 2a. The microcontroller 14 operates on low power and low voltage supplied from the power source 12. The microcontroller 14 allows operation down to 1.8 volts before operation stops. The executable code has a counter 18 and a sine wave generator routine 20 that causes the microcontroller 14 to perform operations such as, for example, generating a twenty-four hour counter 18 that continuously runs and does not need to be synchronized with local time, monitoring time over a twenty-four hour period with the counter 18 and/or altering or modulating a frequency of a sine wave that was generated by the sine wave generator routine 20. The sine wave frequency is altered or modulated over a twenty-four hour period.

When power is first applied to the microcontroller 14, the microcontroller 14 performs a system initialization 22 and then maintains constant operation within a main program loop 24. The functions of the main program loop 24 are described below.

The main program loop 24 of the executable code uses a hardware timer 26 which generates an interrupt each second. The counter 18 is used for counting purposes and may increment seconds at each interrupt of the hardware timer 26. If the counter 18 reaches a count of sixty seconds, the seconds on the counter 18 are cleared to zero and minutes are incremented on the counter 18. If the counter 18 reaches a count of sixty minutes, the minutes of the counter 18 are cleared to zero and hours are incremented on the counter 18. If the counter 18 reaches a count of twenty-four hours, the hours of the counter 18 are cleared to zero, and the counter 18 begins counting from zero seconds to twenty-four hours again. The hardware timer 26 remains active as long as the battery voltage from the power source 12 is above a low voltage state such as, for example, greater than 1.8 volts.

The routine 16 makes a determination at step 28. At step 28, the microcontroller 14 determines if three seconds have elapsed. If three seconds have elapsed, a sensor 30 is used at step 32 to detect if the sensor 30 is located in proximity or close proximity to a living being wearing the magnetic field generator 10. The sensor 30 is coupled to the microcontroller 14. The sensor 30 is in electrical communication with the microcontroller 14.

The sensor 30 makes a determination at step 32 if the sensor 30 is located in proximity or close proximity to the living being wearing the magnetic field generator 10. If the sensor 30 detects that the magnetic field generator 10 is located in proximity or close proximity to the living being at step 32, the microcontroller 14 executes the sine wave generator routine 20 at step 34 and returns to the counter 18 of the main program loop 24. The sine wave generator routine 20 is described below. If the sensor 30 detects that the magnetic field generator 10 is not located in proximity or close proximity to the living being, the main program loop 24 returns to the counter 18. The microcontroller 14 continues to perform operations where time is monitored over a twenty-four hour period with the counter, frequency is altered with the sine wave generator routine, but generation of a magnetic field is discontinued.

If three seconds have not elapsed at step 28, the routine 16 makes a determination at step 36. At step 36, the microcontroller 14 determines if sixty minutes have elapsed. If sixty minutes have not elapsed at step 36, the main program loop 24 returns to the counter 18. If sixty minutes have elapsed at step 36, a determination is made at step 38. At step 38, the microcontroller 14 determines if the number of hours counted by the counter 18 equals twenty-three. If the number of hours counted at step 38 does not equal 23, an index pointer for a sine wave dithering table is incremented at step 40. After step 40, a dither value pointed to by the index pointer is saved in the sine wave generator routine 20 and the main program loop 24 returns to the counter 18 after step 42. If the number of hours counted at step 38 does equal twenty-three, the hour count in the counter 18 is cleared to zero at step 44. After step 44, the index pointer for the sine wave dithering table is cleared to zero at step 46. After step 46, the dither value pointed to by the index pointer is saved in the sine wave generator routine 20 and the main program loop 24 returns to the counter 18.

The sine wave generator routine 20 operates as is described below. At step 48, the microcontroller 14 loads the dither value saved from step 42. Next, at step 50, the microcontroller 14 loads the accumulator or buffer with the least significant bit (“LSB”) of a sixteen bit phase accumulator. Next, at step 52, the microcontroller 14 adds a fractional constant to the accumulator or buffer and stores the LSB in the sixteen bit phase accumulator. At step 54, the microcontroller 14 loads the accumulator or buffer with the most significant bit (“MSB”) of the sixteen bit phase accumulator. Next, at step 56, the microcontroller 14 adds an integer constant and carries the bit to the accumulator or buffer and stores the MSB in the sixteen bit phase accumulator. Next, at step 58, the microcontroller 14 loads an index register with the accumulator or buffer value for sine table indexing. Next, at step 60, the microcontroller 14 loads the accumulator or buffer with the dither value in the sine table pointed to by the index register. Next, at step 62, the microcontroller 14 outputs an eight bit value from the accumulator to a digital to analog converter 64.

The digital to analog converter 64 is coupled to the microcontroller 14. The digital to analog converter 64 is in electrical communication with the microcontroller 14. The digital to analog converter 64 receives a digital output from the microcontroller 14 and converts the digital output to a sine wave voltage which varies from zero volts to the voltage of the power source 12.

As can be seen from step 28 and step 32, the sine wave generator routine 20 is executed every three seconds if the sensor 30 determines that the magnetic field generator 10 is located in proximity or close proximity to the living being wearing the magnetic field generator 10. As can be appreciated by a person of ordinary skill in the art, the sine wave generator routine 20 may be executed at any time from about every two seconds to about every ten seconds at steps 28, 32 and the three second disclosure of FIG. 2a should not be considered a limitation of the invention. The sine wave generator routine 20 creates at least ten cycles at a frequency from about 7.3 hertz to about 8.3 hertz. This frequency range is known as the Schumann Resonance and is also referred to as the Earth's natural frequency. An output from the digital to analog converter 64 and a coil driver 66 then sources current into a coil 68 creating a pulsed sinusoidal magnetic field of an alternating current.

When the magnetic field generator 10 is not in use, the power source 12 does not use battery power to drive the coil 68. To determine if the magnetic field generator 10 is in use, the sensor 30 at step 32 is used to detect whether or not the sensor 30 is located in proximity or close proximity to the living being wearing the magnetic field generator 10. Sensor 30 input is checked every sixty seconds. If the sensor 30 detects that it is located in proximity or close proximity to the living being within the last sixty seconds, the sine wave generator routine 20 is executed at step 34 to drive a current into the coil 68 of the magnetic field generator 10.

If the magnetic field generator 10 is removed and is no longer being worn, the magnetic field generator 10 will be placed in a location where it is not located proximate to the living being that was wearing the magnetic field generator 10. If the sensor 30 detects that it is not located proximate to the living being that was wearing the magnetic field generator 10 after sixty seconds, the microcontroller 14 will not generate a magnetic field and return to the counter 18 of the main program loop 24. The sensor 30 helps extend the lifetime of the battery which is the power source 12.

When an hour is incremented on the counter 18 during operation of the counter 18, the base frequency of the sine wave generator routine is altered with the dither value saved at step 42. When the counter 18 value for hours is at zero which occurs, for example, at midnight or noon, the base frequency is at its lowest value of 7.3 hertz. As the counter 18 increments hours, a constant is added to the base frequency every hour by the dither value saved at step 42. When the counter 18 value for hours is at six which occurs, for example, at six o'clock in the morning or six o'clock in the evening, the base frequency is at its highest value of 8.3 hertz. The process of altering the base frequency of the sine wave generator program mimics the natural diurnal process of the Earth's Schumann Resonance frequency.

The sine wave generator routine 20 uses a sine wave table which provides an eight bit binary value to an output port of the microcontroller 14. This output port is coupled to the digital to analog converter 64 which is an eight bit digital to analog converter 64. The sine wave generator routine 20 generates a pure sine wave tone from a strength of about five milligauss to about fifty milligauss in a frequency range of about 7.3 hertz to about 8.3 hertz from a center frequency of 7.8 hertz. This frequency range is known as the Schumann Resonance frequency range. The Earth's ionosphere alters the frequency of the Schumann Resonance on a daily basis from a frequency of about 7.3 hertz to about 8.3 hertz. The dithering process of the magnetic field generator 10 mimics the natural diurnal process of the Earth's Schumann Resonance field. Certain scientific studies indicate that exposure of living beings to these magnetic fields promote a state of calmness and relaxation. The microcontroller 14 can modulate the frequency that is emitted by the magnetic field generator 10 over time just like the Earth's daily modulation of the Schumann Resonance.

When the digital to analog converter 64 receives a digital output from the microcontroller 14, the digital to analog converter 64 converts the digital output to a sine wave voltage which varies from zero volts to the voltage of the power source 12. The microcontroller 14 is an eight bit lower power microcontroller 14 which drives the digital to analog converter 64. The digital to analog converter 64 is an eight bit digital to analog converter 64. The digital to analog converter 64 is implemented in a passive R2R resistor network to reduce power source 12 current that is supplied to the digital to analog converter 64.

The sensor 30 is responsive to detecting if the magnetic field generator 10 is located in proximity or close proximity to the living being that may be wearing the magnetic field generator 10. If the sensor 30 detects that it is located in proximity or close proximity to the living being wearing the magnetic field generator 10, the microcontroller 14 continues to perform operations wherein time is monitored over a twenty-four hour period with the counter 18 and frequency is altered with the sine wave generator routine 20.

If the sensor 30 detects that it is not located in proximity or close proximity to the living being that was wearing the magnetic field generator 10, the microcontroller 14 continues to perform operations wherein time is monitored over the twenty-four hour period with the counter and frequency is altered with the sine wave generator routine, but discontinues generation of a magnetic field.

The sensor 30 may be of a ball contact type that activates an interrupt input pin on the microcontroller 14 when it detects that it is located in proximity or close proximity to the living being wearing the magnetic field generator 10. As long as the sensor 30 detects that is it located in proximity or close proximity to the living being wearing the magnetic field generator 10, the microcontroller 14 activates the sine wave generator routine 20 to produce a pulsed sine wave current into the circuitry of the magnetic field generator 10 about every two to about every ten seconds. When the living being has removed the magnetic field generator 10 and the sensor detects that it is not located in proximity or close proximity to the living being that had been wearing the magnetic field generator 10 for sixty seconds, the microcontroller 14 ceases pulsing current into the circuitry of the magnetic field generator 10. The ceased pulsing by the microcontroller 14 reduces the current supplied throughout the circuitry of the magnetic field generator 10 which assists in extending battery life of the power source 12. The microcontroller 14 continues to operate the counter 18 at a minimal battery current level. When the sensor 30 detects that it is located in proximity or close proximity to a living being wearing the magnetic field generator 10 again, the microcontroller 14 resumes pulsing current into the coil 68 of the magnetic field generator 10.

The coil driver 66 is coupled to the digital to analog converter 64. The coil driver 66 is in electrical communication with the digital to analog converter 64. The coil driver 66 buffers the high impedance output voltage from the digital to analog converter 64. The coil driver 66 may be an amplifier or a transistor. The coil driver 66 supplies an effective amount of current to the coil 68 to drive a waveform into the coil 68 to generate a magnetic field. The coil 68 generates a pulsed magnetic field having an alternating current with a strength of about five milligauss to about fifty milligauss and is in a frequency range of about 7.3 hertz to about 8.3 hertz.

A current limiter 70 is coupled to the coil driver 66. The current limiter 70 is in electrical communication with the coil driver 66. The current limiter 70 consists of a resistor which limits the current supplied from the coil driver 66 to regulate and establish the strength of the pulsed magnetic field of an alternating current emitted from the coil 68. The coil 68 may be a planar spiral 72, a multi-turn cable 74 or a multi-layer solenoid 76.

The planar spiral coil 72 configuration consists of a flat copper spiral coil chemically etched on a fiberglass or polyimide printed circuit substrate. A magnetic field is generated ninety degrees relative to and equally through the plane of the planar spiral coil 72.

The multi-turn cable 74 configuration consists of multiple conductors that are electrically connected in series within the magnetic field generator 10 circuit board. This electrical coupling converts the multi-turn cable 74 into a series wound coil. A radial magnetic field is generated ninety degrees relative to the cross-section of the conductors.

The multi-layer solenoid 76 configuration consists of multiple turns of a magnetic wire on a bobbin or other supporting structure. The magnetic wire may be American Wire Gauge (“AWG”) number forty wire or smaller. The multi-layer solenoid 76 configuration uses a multi-turn solenoid winding which could contain a highly permeable magnetic material such as, for example, a ferrite rod or laminations of amorphous metallic glasses within the coil 68 in order to increase and create a localized magnetic field. The magnetic field is generated laterally along the axis of the coil 68.

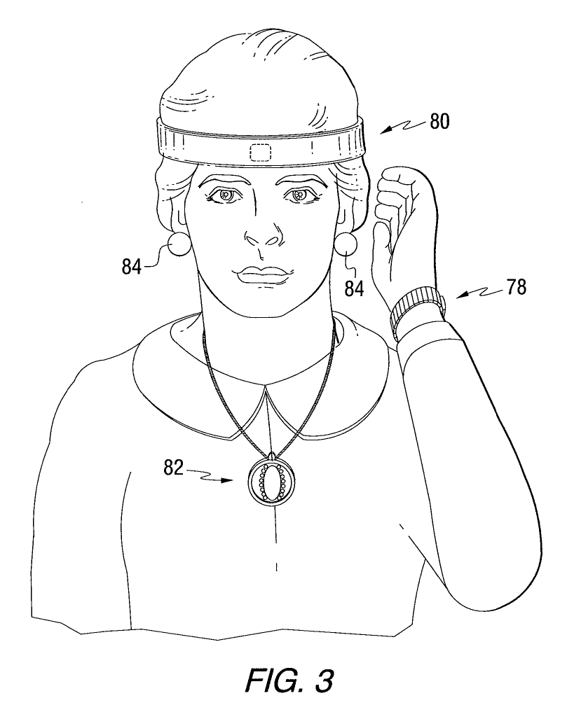

As shown in FIG. 3, the planar spiral 72 configuration may be coupled to a watch 78 or an earring 84. The multi-turn cable 74 configuration may be coupled to a pendent 82 or a headband 80. The multi-layer solenoid 76 configuration may be coupled to the pendent 82 or the headband 80.

The headband 80 could contain a number of the coils 68 such as, for example, a number of planar spirals 72, a number of the multi-turn cables 74, a number of the multi-layer solenoids 76, or combinations thereof to produce various shapes of magnetic fields. The headband 80 could then be placed within an absorbable fabric and used as a sweatband during athletic activity.

A charger/tester 86 may be provided with the magnetic field generator 10. The charger/tester 86 is structured to charge the power source 12 when a rechargeable battery or the like is used as the power source 12. The charger/tester 86 is also structured to detect and optionally display the magnetic field emitted from the magnetic field generator 10.

In an embodiment where a rechargeable power source 12 is used, a transistor switch 88 as shown in FIG. 1 is provided that is controlled by and is in electrical communication with the microcontroller 14. The transistor switch 88 is coupled to the coil 68 and to the power source 12, through current sensing circuit 90 when the magnetic field generator 10 is in a charge mode. Also, during the charging process, the power source 12 voltage level is continuously monitored through a battery voltage sensor 92.

When the magnetic field generator 10 is placed on the charger/tester 86, in charge mode, a current path then exists from the coil 68 to the power source 12. The current sensing circuit 90 produces an analog voltage relative to the level of power source 12 charging current. This voltage is supplied to an analog to digital converter resident within the microcontroller 14 which allows resident executable code to maintain a constant current into power source 12 throughout the charging process.

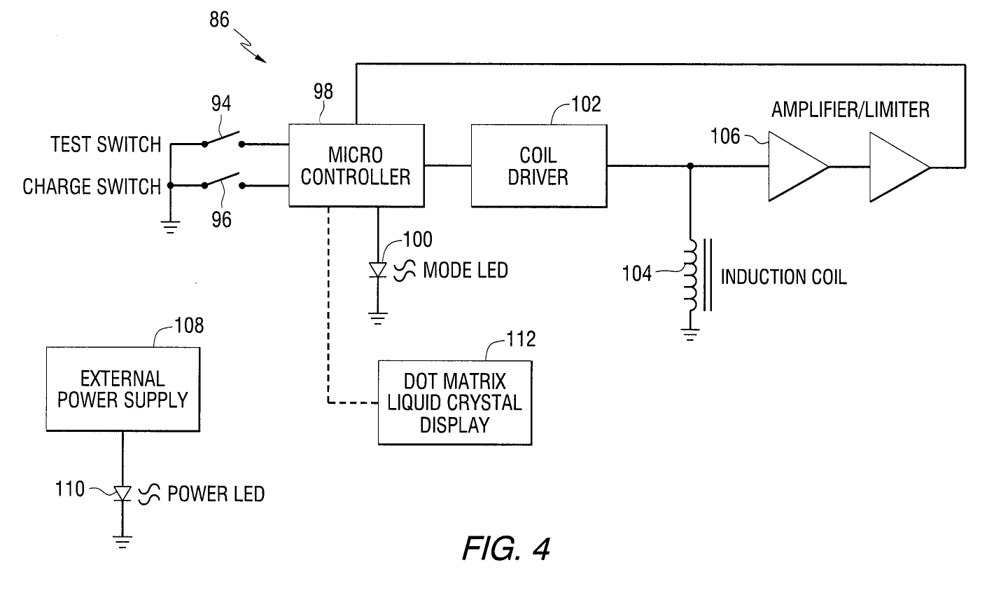

As shown in FIG. 4, the charger/tester 86 is an apparatus that permits charging of the power source 12 within the magnetic field generator 10 and testing of the magnetic field emitted by the magnetic field generator 10. The charger/tester 86 includes an induction coil 104 which is a multi-turn coil with a ferrite core that is supplied with current pulses to create a magnetic field used for power source 12 charging or alternatively used as a magnetic sensing coil to detect and measure the magnetic field emitted by the magnetic field generator 10.

A test switch 94 is in electrical communication with a microcontroller 98 and is used to place the system in a test mode. In the test mode, the magnetic field generator 10 is placed in proximity or close proximity to the induction coil 104. The magnetic field output from the coil 68 is inductively coupled into the induction coil 104. The resultant voltage from this magnetic coupling is amplified by amplifier/limiter 106 that is in electrical communication with the microcontroller 98. The amplifier/limiter 106 provides a square wave output to the microcontroller 98. Executable code resident in the microcontroller 98 then detects and measures the period of the square wave verifying that the magnetic field is within the frequency range of about 7.3 hertz to about 8.3 hertz and illuminates a mode light emitting diode (“LED”) 100 such as green. For example, the mode LED 100 could illuminate a green color to indicate that the magnetic field of the magnetic field generator 10 is within a specified range and that the magnetic field generator 10 is functional.

A charge switch 96 is in electrical communication with the microcontroller 98 and is used to place the system in charge mode. In the charge mode, the microcontroller 98 is in electrical communication with a coil driver 102. The microcontroller 98 supplies switched periodic current pulses to the induction coil 104 creating a pulsed magnetic field that when placed in proximity or close proximity to the magnetic field generator 10 is inductively coupled into the coil 68 of the magnetic field generator 10. The mode LED 100 is then illuminated red to indicate that the magnetic field generator 10 is being charged, for example. A power supply 108 is an external AC power line supply that provides a constant DC voltage to the microcontroller 98, coil driver 102 and the amplifier/limiter 106 for operation. A power light emitting diode (“LED”) 110 is illuminated when the power supply 108 output is supplying power to the system. Other form factors of the charger/tester 86 may include a dot matrix type liquid crystal display 112 that is in electrical communication with the microcontroller 98 to provide a time and/or frequency domain display of the magnetic field emitted by the magnetic field generator 10.

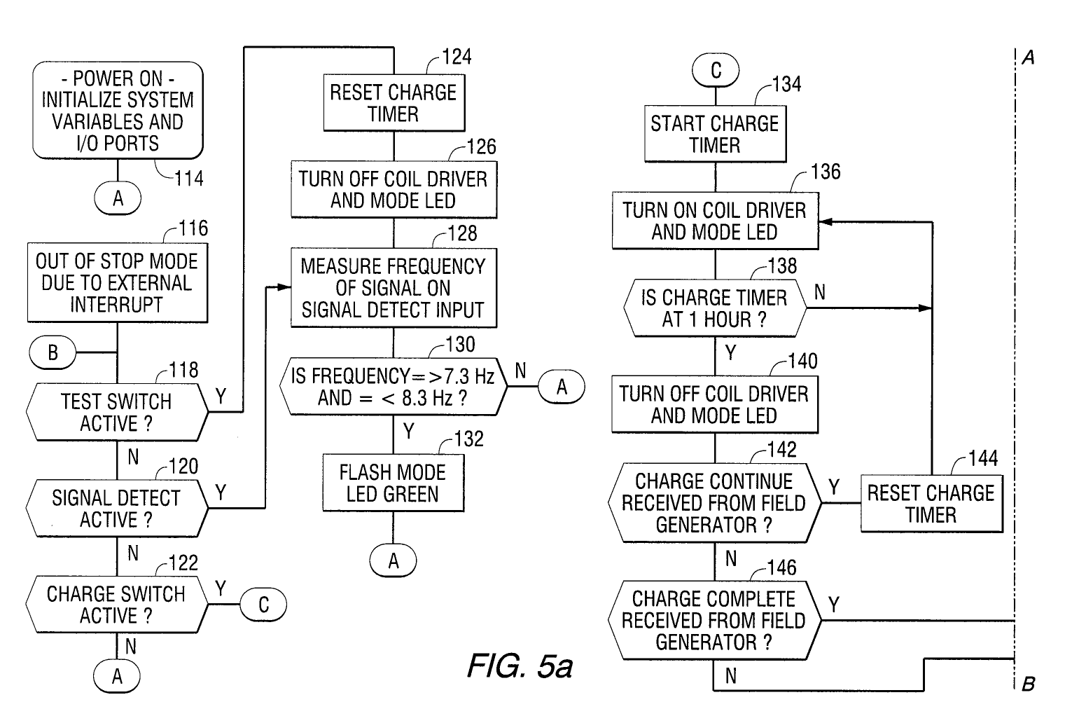

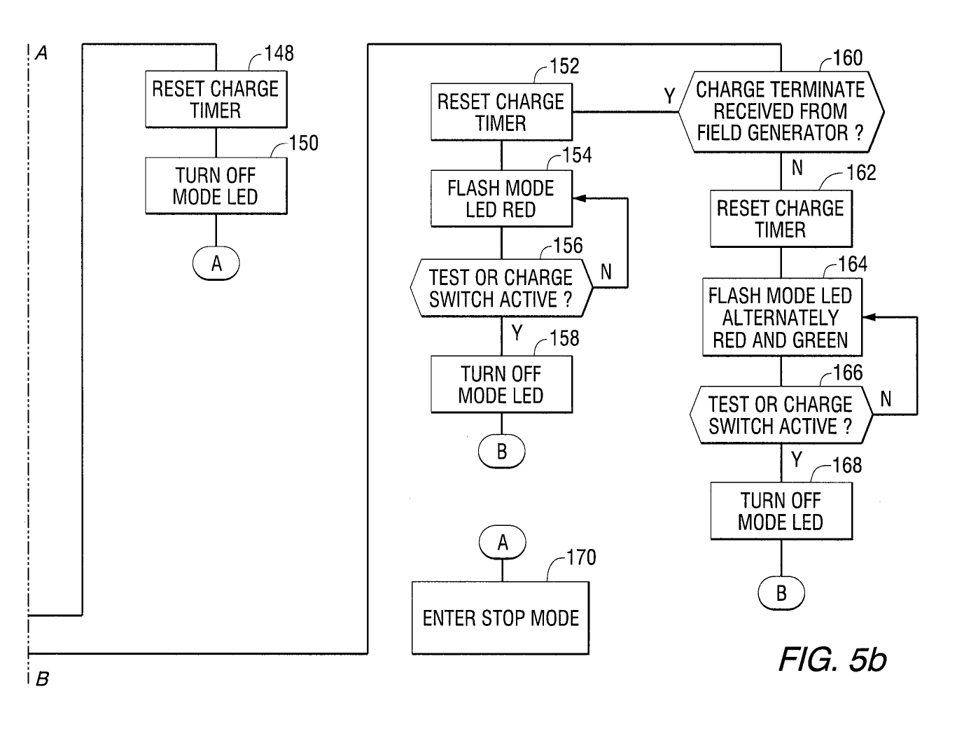

The charger/tester 86 employs the use of the microcontroller 98 that contains executable code that executes the routine shown in FIGS. 5a and 5b and operates on a low voltage provided from the external power supply 108. The charger/tester 86 may operate in a test mode or a charge mode. The executable code monitors the closure of the test switch 94, closure of the charge switch 96 or the output of the amplifier/limiter 106. Upon detection of any of these three inputs becoming active, various code routines are executed to perform a desired function. The charger/tester 86 communicates with the magnetic field generator 10 during the charging process to acknowledge that the magnetic field generator 10 is operational and controlling the charging process. When the power source 12 in the magnetic field generator 10 has been fully charged, the magnetic field generator 10 sends a command to the charger/tester 86 to terminate the charging process.

When power is first applied to the charger/tester 86 and the microcontroller 98, the microcontroller 98 performs a system initialization 114 as shown in FIG. 5a and then enters stop mode 170 as shown in FIG. 5b. The functions of the main program of the charger/tester 86 are described below.

When the microcontroller 98 has entered the stop mode 170, the microcontroller 98 is placed in a low power state that consumes minimum power. After system initialization 114, the microcontroller 98 waits for closure of the test switch 94, closure of the charge switch 96 or an output signal from amplifier/limiter 106 to bring the charger/tester 86 out of the stop mode 170 into an operational mode 116.

A determination is made at step 118. If the test switch 94 is closed, a charge timer is reset at step 124. Next, at step 126, the coil driver 102 and mode LED 100 is turned off. Next, at step 128, the amplifier/limiter 106 measures the frequency emitted by the magnetic field generator 10.

A determination is made at step 130. If the frequency measured by the amplifier/limiter 106 is within a range of about 7.3 hertz to about 8.3 hertz, the microcontroller 90 then flashes the mode LED 100 with a green illumination at step 132 for so long as the magnetic field generator 10 is in proximity or close proximity to the induction coil 104.

If the microcontroller 92 was brought out of the stop mode 170 at the determination step 130 due to a transient signal from the induction coil 104 which was detected by the amplifier/limiter 106 and not determined to be within the frequency range of about 7.3 hertz to about 8.3 hertz or was not of sufficient signal duration, the microcontroller 98 is returned to the stop mode 170.

Alternatively, if the test switch 94 is open at the determination step 118, the main program continues along to make a determination at step 120. If the amplifier/limiter 106 measures a frequency emitted by the magnetic field generator 10 due to an operational magnetic field generator 10 being located in proximity or close proximity of induction coil 104, a continuous pulsed signal will be present at the output of the amplifier/limiter 106 when. The main program then performs the steps 128, 130, 132 as described above. The description of steps 128, 130, 132 is incorporated by reference into this paragraph as if fully set forth herein.

Alternatively, if at the determination step 120, the amplifier/limiter 106 does not measure a frequency emitted by the magnetic field generator 10, the main program continues along to make a determination at step 122. At step 122, if the charge switch 94 is closed, a charge timer is started at step 134. Next, at step 136, the coil driver 102 is activated and the mode LED 100 is illuminated with a red color. The charger/tester 86 then remains in a code loop incrementing the charge timer every sixty seconds. Within the loop between step 136 and step 138, a determination of the value of the charge timer is compared to one hour or sixty minutes at step 138.

If at the determination step 138, the value contained in the charge timer is less than one hour or sixty minutes, the loop between step 136 and step 138 remains active. If at the determination step 138, the value contained in the charge timer is equal to one hour or sixty minutes, the code turns off the coil driver 102 and mode LED 100 off at step 140. The microcontroller 98 then waits for a digital command signal from the magnetic field generator 10 to appear from the output of amplifier/limiter 106. The microcontroller 98 then decodes the incoming data to make a determination at step 142 whether or not the magnetic field generator 10 sent a charge continue command, a charge complete command or a charge terminate command.

If a continue charging command was received, at determination step 142, the charge timer is reset to a value of zero at step 144. The coil driver 102 is turned on and the mode LED 100 is illuminated red at step 136. The code then re-enters a loop of steps 136, 138 and repeats the process described above again.

If a charge continue command was not received from magnetic field generator 10 at the determination step 142, a determination is made at step 146. If a charge complete command is received from the magnetic field generator 10 at the determination step 146, the microcontroller 98 terminates the power source 12 charging process by resetting the charge timer to zero at step 148, turning off the mode LED 100 at step 150 and entering stop mode 170.

After entering the stop mode 170, the magnetic field generator 10 will be producing magnetic wave bursts that will cause the code to flash the mode LED 100 with a green illumination for as long as the magnetic field generator 10 is in proximity or close proximity to the induction coil 104 notifying the user that the power source 12 in the magnetic field generator 10 is now fully charged.

If a charge complete command is not received from the magnetic field generator 10 at the determination step 146, a determination is made at step 160. If a terminate charge command is received from the magnetic field generator at step 160, the charge timer is reset to a value of zero at step 152. The code then enters a loop of steps 154 and 156. At step 154, the mode LED 100 is illuminated with a red color to indicate to the user that the power source 12 in the magnetic field generator 10 is fully charged and will not accept further charging. Step 154 could occur, for example, when a recently charged magnetic field generator 10 is placed on the charger/tester 86, the user activates the charge switch 96 and the charging process is started. A determination is made at step 156. If the test switch 94 or the charge switch 96 is not activated at step 156, the code remains in the loop of steps 154, 156.

If the test switch 94 or the charge switch 96 is activated at step 156, the mode LED 100 is turned off at step 158 and determinations may be made of whether the test switch 94 or the charge switch 96 has been activated at steps 118, 120. Steps 118, 120 have been described above and are incorporated by reference as if fully set forth herein.

If a terminate charge command is not received from the magnetic field generator at step 160, the absence of receiving the command could indicate, for example, that the power source 12 is not accepting a charge or there is a component failure within the magnetic field generator 10 that is preventing the magnetic field generator 10 from issuing a command responsive to when charge pulses are terminated from the charger/tester 86. When the absence of receiving the command occurs at step 160, the charge timer is reset to a value of zero at step 162.

The code then enters a loop of steps 164 and 166. At step 164, the mode LED 100 is illuminated with an alternatively flashing red color and green color. A determination is made at step 166. If the test switch 94 or the charge switch 96 is not activated at step 166, the code remains in the loop of steps 164, 166.

If the test switch 94 or the charge switch 96 is activated at step 166, the mode LED 100 is turned off at step 168 and determinations may be made of whether the test switch 94 or the charge switch 96 has been activated at steps 118, 120. Steps 118, 120 have been described above and are incorporated by reference as if fully set forth herein.

When the charger/tester 86 is in charge mode, the user may activate the test switch 94 at any time which then terminates the charging process and places the system in test mode. The test mode then may perform steps 124, 126, 128, 130 and 132 as described above. The description of steps 124, 126, 128, 130 and 132 is incorporated by reference into this paragraph as if fully set forth herein.

If the test switch 94 is activated during charge mode and the power source 12 in the magnetic field generator 10 has not completed the charge cycle or if the power source 12 in the magnetic field generator 10 is not at a sufficient level of voltage to maintain operation, the magnetic field generator 10 will not produce a magnetic field burst. The omission of the burst will indicate to the user that the charge switch 96 requires re-activation to complete the charging process.

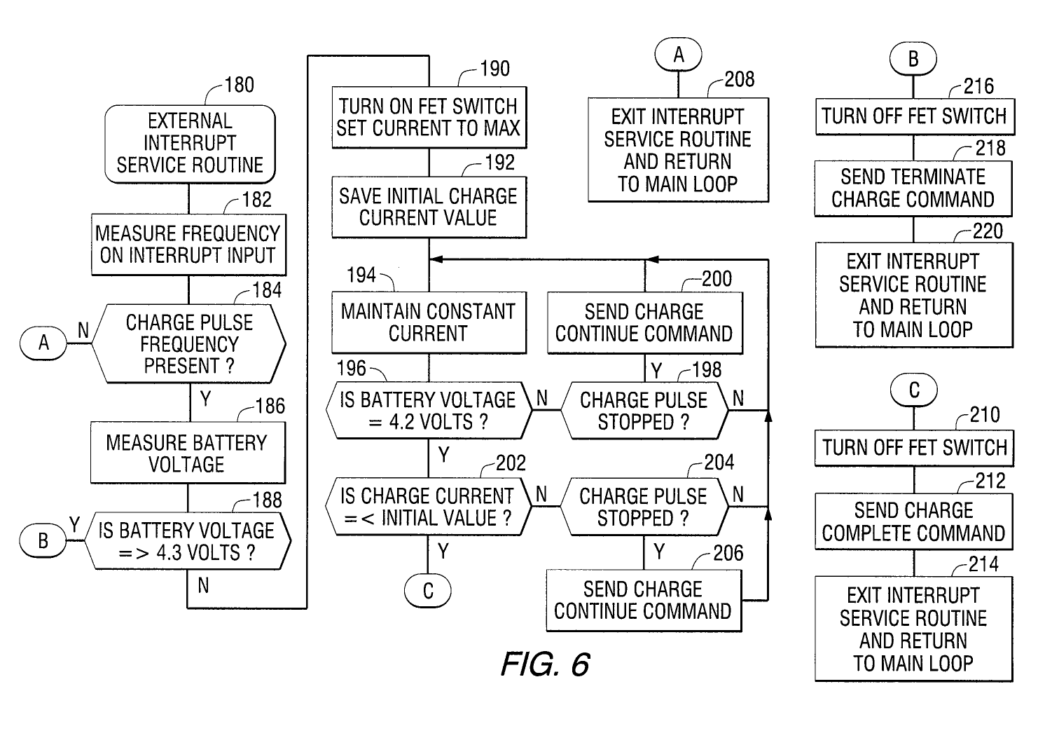

Executable code described in FIG. 6 operates to recharge the power source 12 within the magnetic field generator 10 when it is placed on charger/tester 86 in charge mode. The pulsed magnetic field emitted from induction coil 104 in the charger/tester 86 is a fixed frequency square wave with a duty cycle of about fifty percent. The induction coil 104 is coupled into the coil 68 within the magnetic field generator 10 by induction and when enabled by microcontroller 14 creating a current flow into the power source 12. When the microcontroller 14 in the magnetic field generator 10 detects charge pulses from the coil 68, an external interrupt service routine is executed at step 180. The frequency of the charge pulses are measured at step 182.

A determination is made at step 184. If the pulse frequency is found to be out of range at the determination step 184, the code ceases operation at step 208 and returns to the main loop 24. If the pulse frequency is found to be within the required limit at the determination step 184, the charging process begins.

At the beginning of the charge process, the present value of the power source 12 voltage in the magnetic field generator 10 is measured at step 186. Next, a determination is made at step 188. If the measured voltage is found to be equal to or greater than 4.3 volts at the determination step 188, the charging process is terminated by steps 216, 218 and 220. At step 216, the transistor switch 88 is turned off. At step 218, the magnetic field generator 10 sends a command to the charger/tester 86 to terminate the charging process. At step 220, the external interrupt service routine is terminated and returns to the main loop 24. A voltage at this level would indicate that the power source 12 is already fully charged and, in this condition, it could be hazardous to re-apply current into the power source 12.

If the measured voltage is found to be less than 4.3 volts at the determination step 188, the charging process begins. The transistor switch 88 is then turned on and set to supply maximum charging current at step 190. Maintaining a constant charge current is accomplished by varying the time that the transistor switch 88 is on during the charge pulse supplied from the coil 68 and uses a form of pulse width modulation to vary the charging current to the power source 12. The current sense circuit 90 measures the current supplied to the power source 12 during the charge pulse with its output then being supplied to an analog to digital converter within the microcontroller 10. The initial charge current value is saved at step 192 for later use by the code loop. During the charging process, the power source 12 voltage level is continuously monitored through a battery voltage sensor 92.

When the transistor switch 88 is activated by the microcontroller 14, the battery voltage sensor 92 is also made active which then supplies the present power source 12 voltage from its output to an analog to digital converter within the microcontroller 14. At step 194, a constant current is maintained to the power source 12. A determination is made at step 196. If it is determined that the power source voltage is less than 4.2 volts at step 196, the executable code remains in a loop and performs steps 194, 196, 198 and 200.

At step 196, the executable code loop monitors the voltage which will rise during the charge cycle. As long as the power source 12 voltage is less than 4.2 volts, a constant current to the power source is maintained.

A determination is made at step 198. If charge pulses supplied by the charger/tester 86 are stopped at step 198, a sixty-minute timeout initiated by the microcontroller 98 within the charger/tester 86 has occurred. The timeout provides the opportunity for the magnetic field generator 10 to send a charge continue command at step 200 to the microcontroller 98 within the charger/tester 86 in order to resume the generation of charge pulses.

If there is no response from the magnetic field generator 10, the charger/tester 86 terminates the charging process. The termination would occur if there was a failure of a component within the magnetic field generator 10 and is provided as a safety precaution to avoid overcharging the power source 12 within the magnetic field generator 10. If charge pulses supplied by the charger/tester 86 are not stopped at step 198, the executable code remains in a loop and performs steps 194, 196, 198 and 200.

Near the end of the charging cycle, the power source 12 voltage will reach and stabilize at about 4.2 volts. At this point, the charging current will then begin to decrease until it reaches about ten percent of its original value at the beginning of the charge cycle. The constant charge current loop 194, 196, 198 and 200 will no longer be able to maintain a constant charge current.

When this occurs, a new code loop is entered in steps 202, 204 and 206 which compares the present charge current value to the initial charge current value determined at the start of the charge cycle at step 192. A determination is made at step 202. If the present charge current value is not equal to or less than the initial charge current value at the determination step 202, a determination is made at step 204. If charge pulses supplied by the charger/tester 86 are stopped at step 204, a sixty-minute timeout initiated by the microcontroller 98 within the charger/tester 86 has occurred. The timeout provides the opportunity for the magnetic field generator 10 to send a charge continue command at step 206 to the microcontroller 98 within the charger/tester 86 in order to resume the generation of charge pulses.

If there is no response from the magnetic field generator 10, the charger/tester 86 terminates the charging process. The termination would occur if there was a failure of a component within the magnetic field generator 10 and is provided as a safety precaution to avoid overcharging the power source 12 within the magnetic field generator 10. If charge pulses supplied by the charger/tester 86 are not stopped at step 204, the executable code remains in a loop and performs steps 202, 204 and 206.

If the present charge current value is equal to the initial charge current value at the determination step 202, the power source 12 is considered fully charged and the process is terminated by steps 210, 212 and 214. At step 210, the transistor switch 88 is turned off. At step 212, the magnetic field generator 10 sends a terminate charge command to the charger/tester 86 that the charging process has been completed. At step 214, the external interrupt service routine is terminated and returns to the main loop 24.

Upon the continuation, completion or early termination of the charge cycle, the microcontroller 14 in the magnetic field generator 10 generates a square wave signal through digital to analog converter 64 of the magnetic field generator 10 that appears at the coil 68 as a magnetic field. The magnetic field is then amplified and sent to the microcontroller 98 in the charger/tester 86 for demodulation. The Manchester form of data modulation is used to send digital command data from the magnetic field generator 10 to the charger/tester 86.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended hereto and any and all equivalents thereto.

What is claimed is:

1. A magnetic field generator comprising:
a power source;
a microcontroller in electrical communication with the power source; and
a coil in electrical communication with the microcontroller,
wherein the coil generates a pulsed magnetic wave of an alternating current when a sensor determines that the magnetic field generator is located in proximity to a living being,
wherein the magnetic field generator is structured to be coupled to a device, and
wherein the device is structured to be worn by the living being in proximity to the living being.

2. The magnetic field generator of claim 1, wherein the power source is a low voltage power source and the microcontroller operates at a low voltage.

3. The magnetic field generator of claim 1, wherein the microcontroller contains executable code having a counter and a sine wave generator routine, wherein the executable code causes the microcontroller to perform operations comprising:
monitoring time over a twenty-four hour period with the counter; and
altering a frequency of the sine wave generator routine over the twenty-four hour period by reference to a sine wave table and the time provided on the counter.

4. The magnetic field generator of claim 3, further comprising a sensor in electrical communication with the microcontroller wherein responsive to detecting that the magnetic field generator is located in proximity to the living being, the microcontroller continues to perform operations wherein time is monitored over the twenty-four hour period with the counter and frequency is altered with the sine wave generator routine.

5. The magnetic field generator of claim 3, further comprising a sensor in electrical communication with the microcontroller wherein responsive to detecting that the magnetic field generator is not located in proximity to the living being, the microcontroller continues to perform operations wherein time is monitored over the twenty-four hour period with the counter and frequency is altered with the sine wave generator routine, but discontinues generation of a magnetic field.

6. The magnetic field generator of claim 1, wherein the coil is selected from the group consisting of a planar spiral, a multi-turn cable and a multi-layer solenoid.

7. The magnetic field generator of claim 1, wherein the device is selected from the group consisting of a watch, a pendent, a headband and an earring.

8. The magnetic field generator of claim 1, wherein the magnetic field has a frequency range of about 7.3 hertz to about 8.3 hertz.

9. A method of generating a pulsed magnetic wave to a device to be worn by a living being, the method comprising:
monitoring time over a twenty-four hour period;
generating a pulsed magnetic wave of a certain frequency about every two seconds to about ten seconds when a sensor determines that the device is located in proximity to the living being; and
altering the frequency of the pulsed magnetic wave over a twenty-four hour period.

10. The method of claim 9, further comprising detecting whether or not the device is located in proximity to the living being.

11. The method of claim 10, wherein responsive to detecting that the device is not located in proximity to the living being, the monitoring step is continued, the generating step is discontinued and the altering step is continued.

12. The method of claim 10, wherein responsive to detecting that the device is located in proximity to the living being, the monitoring step is continued, the generating step is continued and the altering step is continued.

13. The method of claim 9, wherein the altering step alters the frequency from about 7.3 hertz to about 8.3 hertz.

14. A magnetic field generator system comprising:
a magnetic field generator comprising:
a rechargeable power source;
a microcontroller in electrical communication with the rechargeable power source; and
a coil in electrical communication with the microcontroller,
wherein the coil generates a pulsed magnetic wave of an alternating current when a sensor determines that the magnetic field generator is located in proximity to a living being,
wherein the magnetic field generator is structured to be coupled to a device,
wherein the device is structured to be worn by the living being in proximity to the living being, and
wherein the magnetic field generator is structured to be in communication with a charger/tester.

15. The magnetic field generator system of claim 14, wherein the microcontroller contains executable code having a counter and a sine wave generator routine, wherein the executable code causes the microcontroller to perform operations comprising:
monitoring time over a twenty-four hour period with the counter; and
altering a frequency of the sine wave generator routine over the twenty-four hour period by reference to a sine wave table and the time provided on the counter.

16. The magnetic field generator system of claim 14, further comprising a sensor in electrical communication with the microcontroller wherein responsive to detecting that the magnetic field generator is located in proximity to the living being, the microcontroller continues to perform operations wherein time is monitored over the twenty-four hour period with the counter and frequency is altered with the sine wave generator routine.

17. The magnetic field generator system of claim 14, wherein the charger/tester is structured to be operable in a charge mode.

18. The magnetic field generator system of claim 14, wherein the charger/tester is structured to be operable in a test mode.

19. The magnetic field generator system of claim 14, wherein the charger/tester is structured to be operable in a magnetic field detection mode.

20. The magnetic field generator system of claim 14, wherein the coil is selected from the group consisting of a planar spiral, a multi-turn cable and a multi-layer solenoid.
